(19) United States
(12) Patent Application Publication
(10) Pub. No.: US 2018/0000035 A1
(43) Pub. Date: Jan. 4, 2018

(54) INBRED CORN LINE FAR614, PLANTS, TISSUES AND SEEDS THEREOF

(71) Applicant: Lee Kent French, Lamberton, MN (US)

(72) Inventor: Lee Kent French, Lamberton, MN (US)

(73) Assignee: Suntava, Inc., Afton, MN (US)

(21) Appl. No.: 14/953,361

(22) Filed: Nov. 29, 2015

(51) Int. Cl. A01H 5/10; A23L 33/105; A01H 1/02; C12N 15/82

(52) U.S. Cl. CPC A01H 5/10; C12N 15/8286; C12N 15/8279; C12N 15/8278; C12N 15/8277; A23L 33/105; C12N 15/8274; C12N 15/8275; A01H 1/02; A23V 2002/00

(57) ABSTRACT

According to the invention, there is provided an inbred corn plant designated FAR614 and its anthocyanins extract. This invention relates to the plants, seeds and tissue cultures of the inbred corn plant FAR614, and to methods for producing a corn plant produced by crossing the inbred corn plant FAR614 with itself or with another corn plant, such as another inbred. This invention further relates to corn seeds and plants produced by crossing the inbred plant FAR614 with another corn plant, such as another inbred, and to crosses with related species. This invention further relates to the inbred and hybrid genetic complements of the inbred corn plant FAR614, and also to the SSR and genetic isozyme typing profiles of inbred corn plant FAR614.

### CROSS REFERENCE TO RELATED APPLICATIONS

This application claims benefit under 35 U.S.C §119(e), to U.S. Patent Provisional Application No. 62/093,580, Docket number 001US005, filed on Dec. 18, 2014 the disclosure of which is herein incorporated by reference in its entirety.

### FIELD DESCRIPTION

### Field of the Invention

The present invention relates generally to the production of corn and particularly to the production of inbred corn line FAR614 having characteristically plant parts and tissues rich in anthocyanins. The invention also relates to the use of the inbred in the production of hybrid corn plants and parts and tissue of these hybrid corn plants.

### BACKGROUND OF THE INVENTION

Maize or corn (Zea mays L.) is a major annual crop species grown for grain and forage. A monocot, maize is a member of the grass family (Gramineae) and bears seeds in female inflorescences (usually called ears) and pollen in separate male inflorescences (usually called tassels).

It is becoming increasingly common practice to grow maize, both in the US and worldwide, almost exclusively by growing hybrid varieties (cultivars). These hybrids are typically produced by seed companies and sold to farmers. On farms, maize hybrids are usually grown as a row crop. Corn is sometimes harvested before maturity, the plants may be chopped and placed in storage where the chopped forage undergoes fermentation to become silage for livestock feed. When the plants are left to reach maturity, the seeds are harvested as grain. The grain may be directly fed to livestock or transported to storage facilities. From storage facilities, the grain is transported to be used in making an extremely large number of products, including food ingredients, pigments, snacks, pharmaceuticals, sweeteners, and other products.

Plants that have been self-pollinated and selected for type over many generations become homozygous at almost all gene loci and produce a uniform population of true breeding progeny, a homozygous plant. A cross between two such homozygous plants produces a uniform population of hybrid plants that are heterozygous for many gene loci. Conversely, a cross of two plants each heterozygous at a number of loci produces a population of hybrid plants that differ genetically and are not uniform. The resulting non-uniformity makes performance unpredictable.

The development of uniform corn plant hybrids requires the development of homozygous inbred plants, the crossing of these inbred plants, and the evaluation of the crosses. Pedigree breeding and recurrent selection is examples of breeding methods used to develop inbred plants from breeding populations. Those breeding methods combine the genetic backgrounds from two or more inbred plants or various other broad-based sources into breeding pools from which new inbred plants are developed by selfing and selection of desired phenotypes. The new inbreds are crossed with other inbred plants and the hybrids from these crosses are evaluated to determine which of those have commercial potential.

Types of hybrids include single-cross, three-way, and double-cross. Single-cross hybrids are the F₁ progeny of a cross between two inbred lines (inbreds), e.g., A*B, in which A and B are inbreds. Three-way hybrids are the first generation progeny of a cross between a single-cross hybrid typically used as the female and an inbred, e.g., A*B) (C, in which A*B is a single-cross hybrid of inbreds A and B and C is another inbred. Double-cross hybrids are the first generation progeny of a cross between two single-cross hybrids, e.g., A*B) (C*D, in which A*B and C*D are single-cross hybrids of inbreds A and B and C and D, respectively. In the U.S., single-cross hybrids currently occupy the largest proportion of the acreage used in maize production. As will be shown below, maize inbreds are assemblages of true breeding, homozygous, substantially identical (homogeneous) individuals. Single-cross hybrids are both homogeneous and highly heterozygous and are not true breeding. Three-way and double-cross hybrids are less homogeneous, but are nonetheless highly heterozygous and not true breeding as well. Hence, the only way of improving hybrids is improving component inbreds thereof. Improving maize inbreds involves procedures and concepts developed from the discipline.

In some instances, where several inbred lines are planted close together, plants exhibiting qualities that are superior to those intended may appear in the lot due to incomplete detasseling or other accidental cross pollinations. These plants are then harvested and subjected to the breeding methods herein discussed, e.g., selfing, backcrossing, hybrid production, crosses to other hybrids, inbreds, populations, and the like to produce a new inbred line such as the case of inbred line FAR614.

Some of the most sought after varieties of corn are those with intensely colored plant parts. There is a continuing need to develop better varieties of corn plants with increased and more dependable pigment and grain and stover yields. Moreover, heat and drought stress and continually changing insect pests and disease pathogens present hazards to farmers as they grow maize hybrids. Thus, there is a continual need for maize hybrids which offer higher grain yields in the presence of heat, drought, pathogens and insects.

In accordance with the present invention and in addition to the foregoing agronomic traits, an important characteristic of inbred lines and hybrid plants obtained using the inbred line of this invention involves pigmentation, such as anthocyanins in different parts of the plant.

### SUMMARY OF THE INVENTION

By means of the present invention, there is provided an inbred line of corn having plant parts and tissues with high concentrations of anthocyanins, which is not common in known corn plants used commercially today. These desirable attributes are characterized by inbred corn line such as FAR614

One aspect of the invention involves the plant parts and tissue of this inbred and the use of the plant parts and tissue of this inbred in the extraction and use of the purple plant pigments associated with this inbred and its hybrids.

Another aspect of the invention involves a process for producing seed of an inbred corn line including self-pollinating the inbred FAR614, then harvesting seed. The invention also involves the seed produced by the process of inbreeding the inbred corn lines obtained from crossing FAR614 with other inbreds.

In addition, the invention involves a process for producing a hybrid corn plant utilizing the inbred corn line FAR614. The process involves crossing a first inbred line with a non-identical second inbred line to produce a hybrid corn seed. The hybrid corn seed is harvested and grown to produce a hybrid plant including characteristics of FAR614. The invention also contemplates a hybrid corn plant produced by this process and the hybrid corn seed produced by the process.

An important aspect of the inbred and hybrid corn plants associated with the present invention has to do with the inherent stable color expression present in the plant parts and tissues such as the husk, inflorescences, stem, stalks and leaves which differ dramatically from those of standard yellow or white corn. These tissues, especially after flowering, have a deep red to purple color which is also expressed inside the stem tissue. Even the crown roots show a much higher content of anthocyanins as compared to standard corn varieties. The husk leaves surrounding the ear also appear different in phenotype and anthocyanin concentration not found in the husks of standard or regular corn. This phenomenon is characterized by a red to purple color which intensifies after anthesis. Accordingly, intense pigmentation is present in tissues of the inbred and hybrid of this invention such as, without limitation, inflorescences, husks, cobs, stems, and grain. Consequently, the stover and grain of the inbred of this invention and their hybrid have economic value above that normally present due to the intense pigmentation present. The deep coloration manifested by the inbred of this invention and their hybrid is indicative of high concentrations of pigments, such as anthocyanins, in these tissues.

In the same manner, the grain expresses a deep red to purple color noticeable about two to three weeks after flowering. At physiological maturity, all grains are a complete deep red to purple hue. The ear of this inbred and its hybrids shows a very deep red to pink and purple phenotypic expression which reaches its highest concentration as the grain approaches harvest time.

The FAR614 purple corn inbred and hybrids in other respects perform in a manner similar to other standard corn hybrids grown in a field. These other aspects have been found to be acceptable.

All publications cited herein are hereby incorporated by reference.

### DETAILED DESCRIPTION

Consumers are increasingly rejecting the addition of exogenous chemicals to the food products they consume. One tradition that the consumers are particularly objecting to is the addition of artificial colorants to food. These same consumers do not however object to these colorants when they are extracted from plants. One plant species offering promise in terms of quantity (or yield) and quality (various colors) of such colorants is maize. Accordingly, the inbreds of this invention combine desirable and productive agronomic traits with high yields of extractable pigments.

### Incorporation of Single Nucleotide Polymorphism (SNP) Genotyping Results

One copy of SNP genotyping table named: FAR614SNPtable which was created, on CD-ROM on Dec. 7, 2014 and filed with the provisional application No. 62/093,580, Docket number 001US005 is incorporated herein by reference

### Grain and Stover Production

This invention is contemplated to include producing stover and grain when hybrids with FAR614 as a parent are grown. Typically seed of these hybrids is planted in soil with adequate moisture to support germination, emergence, and subsequent growth and development. Alternatively, soil moisture is added by irrigation. Normal cultural practices to achieve proper soil fertility and manage weeds, insects, and diseases may be undertaken during the growing season as necessary. These cultural practices are known to persons of skill in the art and vary widely according to particular geographic regions, grower preferences, and economic considerations. The corn plants may be chopped for silage, typically when the developing grain is at the half-milk stage. The grain is harvested when physiologically mature, usually with combines, then dried to a moisture content sufficiently low for storage. The grain may then be used for feed, food, and industrial purposes, examples of which are disclosed herein.

### Derivation

This invention is considered to include processes of developing derived (introgressed) maize inbred lines and plants, seeds, and parts resulting thereof. Processes of developing derived inbred lines include those processes, wherein single genes or alleles or some small plurality of genes or alleles are introgressed into FAR614, resulting in a derived inbred which expresses the introgressed gene(s) or allele(s) (i.e. trait(s)), but otherwise retains the phenotype and genotype of FAR614 described herein. Examples of introgressed genes or alleles include insect or disease resistance, genes from other maize plants, or alleles or genes originating from other species. Non-limiting examples of these genes or alleles are disclosed in Coe et al., “The Genetics of Corn,” IN Corn and Corn Improvement, G. F. Sprague and J. W. Dudley, Editors, American Society of Agronomy, Madison, Wis. (1988). Other nonlimiting examples of genes or alleles which might be introgressed into the present invention are disclosed herein below. Methods of introgression may include such protocols as backcrossing, tissue culture to induce somoclonal variation, impaling plant cells with needle-like bodies, use of indeterminate gametophyte, anther culture, and transformation.

Backcrossing protocols are disclosed, e.g in above-referenced F. N. Briggs and P. F. Knowles, Introduction to Plant Breeding, Reinhold Publishing Company, New York (1967), R. W. Allard, Principles of Plant Breeding, Wiley and Sons, New York (1960), N. W. Simmonds, Principles of Crop Improvement, Longman Group, Ltd., London (1979); and J. M. Poehlman, Breeding Field Crops, 2d Ed., AVI Publishing Co., Inc. Westport, Conn. (1979). Use of indeterminate gametophyte-facilitated (ig1) introgression of cytologically inherited traits is disclosed by, e.g., J. L. Kermicle, “Androgenesis Conditioned by a Mutation in Maize,” Science 166: 1422-1424 (1969).

Isolated microspore, anther culture and regeneration of fertile maize plants are disclosed in U.S. Pat. No. 5,445,961 to Genovesi et al. Introgression protocols using anther culture are disclosed, e.g., by Barnabas et al., “Ultrastructural Studies on Pollen Embryogenesis in Maize (Zea mays L)”, Plant Cell Rep. 6: 212-215 (1987); Dieu et al., “Further Studies of Androgenetic Embryo Production and Plant Regeneration From In Vitro Cultured Anthers in Maze (Zea mays L.),” Maydica 31: 245-259 (1986); Pace et al., “Anther Culture of Maize and the Visualization of Embryogenic Microspores by Fluorescent Microscopy,” Theor. Appl. Genet. 73: 863-869 (1987); Petolino et al., “Anther Culture of Elite Genotypes of Maize,” Crop Sci. 26: 1072-1074 (1986); and Tsay et al., “Factors Affecting Haploid Plant Regeneration from Maize Anther Culture,” J. Plant Physiol. 126: 33-40 (1986).

Exemplary transformation protocols are disclosed, e.g., by U.S. Pat. No. 5,302,523 to Coffee et al. (transformed maize via needle-like bodies); U.S. Pat. No. 5,384,253 to Krzyzek et al. (electroporation); U.S. Pat. No. 5,371,003 to Murray et al. (transformation via tissues within horizontal electrophoresis gel in the presence of non-pulsed electric current); U.S. Pat. No. 5,591,616 to Hiei et al. (Agrobacterium-mediated transformation); U.S. Pat. No. 5,569,597 to Grimsley et al. (Agrobacterium-mediated maize transformation); U.S. Pat. No. 5,877,023 to Sautter et al. (microprojectile-facilitated transformation); U.S. Pat. No. 5,736,369 to Bowen et al. (microprojectile-facilitated transformation); U.S. Pat. Nos. 5,886,244 and 5,990,387 to Tomes et al. (microprojectile-facilitated transformation); U.S. Pat. No. 5,776,900 to Shillito et al. (regeneration of maize protoplasts transformed with electroporation and polyethylene glycol (PEG)); U.S. Pat. Nos. 5,767,367 and 5,792,936 to Dudits et al. (regeneration of PEG-transformed protoplasts of auxin-autotrophic maize genotype); U.S. Pat. Nos. 5,780,708 and 5,990,390 to Lundquist et al. (fertile, microprojectile-facilitated transgenic maize plants expressing dalapon resistance); U.S. Pat. Nos. 5,780,709 and 5,919,675 to Adams et al. (microprojectile- and electroporation-facilitated maize transformants); U.S. Pat. No. 5,932,782 to Bidney (microprojectile-delivered Agrobacterium); U.S. Pat. No. 5,981,840 to Zhao et al. (Agrobacterium-transformed maize); and U.S. Pat. No. 5,994,624 to Trolinder et al. (maize transformation via recombinant Agrobacterium DNA injected into plant tissues via needleless injection device). An exemplary transformation protocol is more fully disclosed herein below.

### Further Uses

This invention is also contemplated to include processes or methods of producing a maize plant by crossing a first parent maize plant with a second parent maize plant in which the first or second parent maize plant is the inbred maize line FAR614. Moreover, both the first and second parent maize plants may include the inbred maize lines FAR614.

This invention is also directed to processes or methods of producing FAR614 derived maize plants or an inbred maize plant with FAR614 as a parent in at least one of the initial breeding crosses accomplished by crossing inbred maize line FAR614 with a second maize plant and growing the progeny seed. The method may further include repeating crossing and growing the FAR614-derived plants until the substantial genotype of FAR614 is recovered. Thus, any methods using the inbred maize line FAR614 are contemplated to be within the scope of this invention, e.g., selfing, backcrossing, hybrid production, crosses to other hybrids, inbreds, populations, and the like. All plants produced using inbred maize line FAR614 as a parent are contemplated to be within the scope of this invention, including plants derived from inbred maize line. It should be further understood that inbred maize lines FAR614 can, through routine manipulation known to skilled persons in the art, be produced in a male-sterile form and that such embodiments are contemplated to be within the scope of the present invention as well.

As used herein, the term “plant” includes whole or entire plants and parts thereof. Such exemplary plant parts may include plant cells, plant protoplasts, plant cell tissue cultures, plant calli, plant clumps, plant cell suspension cultures, and plant protoplasts. Also included within the definition of the term “plant” are plant cells present in plants or parts of plants, e.g., zygotes, embryos, embryonic organs, pollen, ovules, flowers, seeds, ears, cobs, leaves, husks, stalks, sheaths, roots, root tips, anthers, and silks.

### Tissue Culture of Maize

Regeneration of maize plants by tissue culture methods is now an exercise requiring only routine experimentation to a person skilled in the art. For example, Duncan et al. (Planta 165:322-332 (1985)) reported 97% of the plant genotypes cultured produced calli capable of plant regeneration. Plants were regenerated from 91% of the calli from another set of inbreds and hybrids in a subsequent experiment. Songstad et al., (Plant Cell Reports, 7:262-265 (1988)) reported several media additions enhancing regenerability of callus of two inbred lines. Other published reports also indicated “nontraditional” tissues capable of producing somatic embryogenesis and plant regeneration. For example, Rao, et al. (Maize Genetics Cooperation Newsletter, 60:64-65 (1986)) reported somatic embryogenesis from glume callus cultures. Conger, et al. (Plant Cell Reports, 6:345-347 (1987)) reported somatic embryogenesis from tissue cultures of maize leaf segments. Thus, it is clear that the state of the art is such that these tissue culture methods of obtaining regenerated plants are routinely used with very high rates of success.

Maize tissue culture is described generally in European Patent Application, Publication 160,390 and with respect to inbred line B73 in U.S. Pat. No. 5,134,074 to Gordon et al. Maize tissue culture procedures are also described by U.S. Pat. No. 4,581,847 to Hibberd et al., by Kamo et al. “Establishment and Characterization of Long-Term Embryonic Maize Callus and Cell Suspension Cultures,” Plant Science 45: 111-117, by Vasil et al., “Plant Regeneration from Friable Embryonic Callus and Cell Suspension Culture of Zea mays L.,” J. Plant Physiol. 124:399-408 (1986), by Green et al., “Plant Regeneration in Tissue Culture of Maize,” Maize for Biological Research (Plant Molecular Biology Association, Charlottesville, Va. 1982, at 367-372) and by Duncan, et al., “The Production of Callus Capable of Plant Regeneration from Immature Embryos of Numerous Zea Mays Genotypes,” 165 Planta 322-332 (1985). Thus, another aspect of this invention is to provide cells, which undergo growth and differentiation and subsequently produce maize plants with the physiological and morphological characteristics of inbred maize line FAR614.

Somaclonal variation within inbred lines which have undergone tissue culture and regeneration have been reported by Edallo et al. (“Chromosome Variation and Frequency of Spontaneous Mutants Associated With In Vitro Culture and Plant Regeneration in Maize,” Maydica 26: 39-56 (1981)); McCoy et al. (“Chromosome Stability in Maize (Zea Mays L.) Tissue Culture and Sectoring in Some Regenerated Plants,” Can. J. Genet. Cytol. 24: 559-565 (1982)), Earle et al. (“Somaclonal Variation in Progeny of Plants From Corn Tissue Culture,” pp 139-152, IN R. R. Henke et al. (ED.) Tissue Culture in Forestry and Agriculture, Plenum Press, N.Y. (1985)); and Lee et al. (“Agronomic Evaluation of Inbred Lines Derived From Tissue Cultures of Maize,” Theor. Appl. Genet. 75: 841-849 (1988)). Hence, genetic variation and derived lines may be developed from this invention by tissue culture protocols.

The utility of inbred maize line FAR614 also extends to crosses with other species. Suitable species will be of the family Gramineae, and especially genera such as Zea, Tripsacum, Coix, Schlerachne, Polytoca, Chionachne, and Trilobachne, of the tribe Maydeae. Potentially suitable for crosses with inbred maize line FAR601 or FAR045 may be the various varieties of grain sorghum, Sorghum bicolor (L.) Moench. or other species within the genus Sorghum.

### Transformation

Molecular biological techniques now allow genes encoding specific protein products to be isolated and characterized. It has long been viewed as advantageous to modify maize plant genomes to contain and express foreign genes, or additional, or modified versions of native or endogenous genes (perhaps driven by different promoters) to alter traits of a plant in a specific, directed manner. Such foreign, additional and/or modified genes are referred to herein collectively as “transgenes” and several methods for producing transgenic plants have been developed. Accordingly, embodiments of this invention also include derived inbreds which are transformed versions of inbred maize line FAR614.

Thus, this invention is contemplated to include transformed, therefore derived, embodiments of inbred maize line FAR614. In another embodiment, the biomass of interest is the vegetative tissue of inbred maize line FAR614. In yet another embodiment, the biomass of interest is grain (seed). For transgenic plants, a genetic map can be generated, primarily via conventional Restriction Fragment Length Polymorphisms (RFLP), Polymerase Chain Reaction (PCR) analysis, Random Amplified Polymorphic DNA (RAPD), Amplified Fragment Length Polymorphisms (AFLP), Single Nucleotide Polymorphisms (SNP), and Simple Sequence Repeats (SSR), which identify the approximate chromosomal location of the integrated DNA. For exemplary methodologies in this regard, see Glick et al., Methods in Plant Molecular Biology and Biotechnology, 269-284 (CRC Press, Boca Raton, 1993). Map information concerning chromosomal location is useful for proprietary protection of a given transgenic plant. Hence, if unauthorized propagation occurs and crosses of the present inbred are made to other germplasm, the map of the integration region can be compared to similar maps of suspect plants, thereby determining whether the suspect plants have a common parentage with the subject plant. Map comparisons require hybridization and subsequent RFLP, PCR, SSR, RAPD, AFLP, SNP and/or sequencing, all of which are known techniques.

Agronomic genes can be expressed in the transformed plants of this invention. More particularly, plants of this invention can be transformed, or otherwise derived, to express various phenotypes of agronomic interest. Exemplary genes implicated in this regard include, but are not limited to, those categorized below.

### Maize Transformation Methods

Plant transformation methods contemplated to transform the inbred of this invention include biological and physical plant transformation protocols. See, e.g., Miki et al., “Procedures for Introducing Foreign DNA into Plants” IN Methods in Plant Molecular Biology and Biotechnology, B. R. Glick and J. E. Thompson, Eds. (CRC Press, Inc., Boca Raton, 1993) pages 67-88; Gruber et al., “Vectors for Plant Transformation” in Methods in Plant Molecular Biology and Biotechnology (expression vectors and in vitro culture methods for plant cell or tissue transformation and regeneration of plants); and B. R. Glick and J. E. Thompson, Eds., CRC Press, Inc., Boca Raton, (1993) pages 89-119 (expression vectors and in vitro culture methods for plant cell or tissue transformation and regeneration of plants).

Transformation methods known in the art may be used to produce transgenic derived inbred lines of this invention. These transgenic inbred lines may then be crossed with another (non-transformed or transformed) inbred line to produce a transgenic hybrid maize plant. Alternatively, a genetic trait introgressed into a maize line using the foregoing transformation protocols may be transferred to another line using traditional backcrossing techniques known to the plant breeding art, e.g., backcrossing an engineered trait from a public, non-elite line into an elite line, or from a hybrid maize plant with a foreign transformed gene into an inbred line not containing that gene. As used herein. “crossing” can refer to a single cross or to the process of backcrossing.

### 14. INDUSTRIAL APPLICABILITY

Maize is used as human food, livestock feed, as raw materials in industry, and as a source of pigments. The food uses of maize, in addition to human consumption of maize kernels, include products of the dry-milling and wet-milling industries, as well as pigments for colorants. The principal products of maize dry milling are grits, meal and flour. The maize wet-milling industry provides maize starch, maize syrup, and dextrose for food use. Maize oil is recovered from maize germ, which is a by-product of both the dry-milling and wet-milling industries.

Hence, the seed of inbred maize line FAR614, the plant produced from the inbred seed, the hybrid maize plant produced from the crossing of the inbred, hybrid seed, and various parts of the hybrid maize plant and transgenic versions of the foregoing, can be utilized for human food, livestock feed, as a raw material in industry, or as a source of extracted pigments.

As previously indicated, the present invention relates to inbred lines of corn, including importantly plant parts and tissue of these inbred lines of corn, seed for such inbred lines, the use of the inbreds to produce hybrid corn plants, hybrid corn plants obtained using at least one of the inbreds as a parent and parts and tissue of these hybrid corn plants.

As used herein, the term “inbred,” “inbred line” or “inbred lines” means a group of plants from a common ancestry which are essentially homozygous and which are true breeding, i.e., uniform and stable with respect to all of their agronomically important characteristics. In preferred embodiments, the inbred lines have the characteristics of the lines designated as FAR614. Generally, the preferred seeds of inbred corn lines in accordance with the invention, have the characteristics of the seeds of each of the designated lines.

Anthocyanins are plant-based polyphenolic pigments belonging to the class of molecules termed flavonoids. Consequently, anthocyanins are a very diverse group of compounds with a basic structure: anthocyanidin glycosidically linked to carbohydrate moieties and/or acyl groups. Anthocyanins commonly found in corn may include Cyanidin 3-glucoside, Cyanidin 3-(6″-malonylglucoside), Cyanidin 3-(6″-ethylmalonylglucoside), Cyanidin 3-(6″-dimalonylglucoside) (and other derivatives); Pelargonidin 3-glucoside. Pelargonidin 3-(6-malonylglucoside), Pelargonidin 3-(6″-ethylmalonylglucoside) (and other derivatives); Peonidin 3-glucoside, Peonidin 3-(6″-malonylglucoside), Peonidin 3-(6″-ethylmalonylglucoside) (and other derivatives); Cyanidin 3-galactoside (and derivatives); Cyanidin 3-rutinoside (and derivatives); and Petunidin 3-glucoside (and derivatives).

Anthocyanins are widely found in flowering plants, such as corn. These compounds are typically water soluble and non-toxic, displaying a range of colors from orange, bright red/purple to blue. Anthocyanins are polar molecules and, therefore, are more soluble in polar solvents. Solubility also depends on various conditions such as pH, temperature, type of solvent, and concentration. Extraction may use solvents such as water, methanol, ethanol, or mixtures thereof, optionally acidified with an acid (e.g., between about 0.001% and 0.01% HCl or citric acid).

Other useful compounds may be present in extracts from the inbreds of this invention and their hybrid, such as other flavonoids, phenolic acids, and carbohydrates. Exemplary flavonoids include (−)-Epicatechin, (−)-Epicatechin 3-gallate, (−)-Epigallocatechin, (−)-Epigallocatechin 3-gallate, (+)-Catechin, (+)-Gallocatechin. Exemplary phenolic acids include Ferulic acid and derivatives, Quercetin and derivatives, P-coumaric acid and derivatives, Protocatechuic acid and derivatives, Vanillic acid and derivatives, Hesperitin and derivatives, Hydroxycinnamic acid and derivatives, Gallic acid and derivatives. A nonlimiting recital of carbohydrates would include common sugars such as arabinose, rhamnose or galactose and/or with acylating acids.

### Description of Maize Inbred FAR614

The inbred corn line FAR614, as indicated, is substantially homozygous and can be reproduced by planting seed of the line, growing the resulting corn plants under self-pollination or sibbing with adequate isolation, and harvesting the resulting seed using techniques familiar to those of skill in the art. A hybrid of FAR614 such as seen in table 2 herein, with another inbred (inbred 1) can be produced by growing the two inbreds in proximity, then detasseling one of the inbreds in this invention. Alternately, other methods of pollen control as more fully described herein can be used as well.

The inbred and its hybrids has shown uniformity and stability within the limits of environmental influence for each of the traits. The inbred is described in Table 1.

TABLE 1
Morphological Characteristics of Inbred FAR614

| UPOV | Characteristic | FAR614 |
|---|---|---|
| 1 | type | 2 (Dent) |
| 2 | Region where best adapted in the USA | 2 (North central) |
| 3 | Maturity | |
| | Days from emergence to 50% of plants | 84 |
| | in silk | |
| | Days from 50% silk to harvest at 25% | 48 |
| | kernel moisture | |
| 4 | Plant | |
| | Height (to tassel tip) (cm) | 179 |
| | Length of top ear internode (cm) | 17 |
| | Ear height (to base of top ear) (cm) | 58 |
| | Number of tillers 1: none; 2: 1-2; 3: 2-3 | 2 |
| | 4: >3 | |
| | Number of ears per stalk 1: single; 2: | 2 |
| | slight two ear tendency; 3: strong two | |
| | ear tendency; 4: three ear tendency | |
| | Cytoplasm type 1: normal; 2: “r”; 3: “s”; | 1 |
| | 4: “c” | |
| | Stalk color 1: yellow, 2: pink; 3: red; 4: | 4 |
| | purple; 5: green | |
| 5 | Leaf | |
| | Color 1: light green; 2: medium green; | 4 with purple midrib |
| | 3: dark green; 4: very dark green | |
| | Angle from stalk (upper half) 1: <30°; 2: | 2 |
| | 30° to 60°; 3: >60° | |
| | Sheath pubescence 1: light; 2: medium; | 2 |
| | 3: heavy | |
| | Marginal waves 1: none; 2: few; 3: | 2 |
| | many | |
| | Longitudinal creases 1: absent; 2: few; | 2 |
| | 3: many | |
| | Width | |
| | Widest point of ear node leaf (cm) | 9 |
| | Length ear node leaf (cm) | 69 |
| | Number of leaves per mature plant | 10 |
| 6 | Tassel | |
| | Number of lateral branches | 15 |
| | Branch angle from central spike 1: <30°; | |
| | 2: 30°-40°; 3: >45° | |
| | Peduncle length (cm) | 6 |
| | Pollen shed 1: light; 2: medium; 3: | 2 |
| | heavy | |
| | Anther color 1: yellow, 2: pink; 3: red; | 1 |
| | 4: purple; 5: green | |
| | Glume color 1: yellow, 2: pink; 3: red; | 4 |
| | 4: purple; 5: green | |
| 7 | Ear (husked except when stated) | |
| | Length (cm) | 13 |
| | Weight (gm) | 69 |
| | Midpoint diameter (mm) | 35 |
| | Kernel rows | |
| | 1: indistinct; 2: distinct | 2 |
| | Number | 19 |
| | 1: strait; 2: slightly curved; 3: spiral | 1 |
| | Silk color 1: green; 2: pink; 3: salmon; | 4 |
| | 4: red; 5: other (Green-yellow) | |
| | Husk color (fresh) 1: light green; 2: dark | 5 |
| | green; 3: pink; 4: red; 5: purple; 6: other | |
| | Husk color (dry) 1: light green; 2: dark | |
| | green; 3: pink; 4: red; 5: purple; 6: other | |
| | Husk extension (harvest stage) 1: short | 2 |
| | (ears exposed); 2: medium (barely | |
| | covering ear); 3: long (8-10 cm beyond | |
| | ear tip); 4: very long (>10 cm) | |
| | Husk leaf 1: short (<8 cm); 2: medium | 2 |
| | (8-15 cm); 3: long (>15 cm) | |
| | Shank (cm) | 10 |
| | Number of internodes | 9 |
| | Position at dry husk stage 1: upright; 2: | 2 |
| | horizontal; 3: pendant 1: slight | |
| | Taper 1: slight; 2: average; 3: extreme | 2 |
| | Drying time (unhusked ear) 1: slow; | 2 |
| | 2: average; 3: fast | |
| 8 | Kernel | |
| | Length (mm) (from mid-ear point) | 7 |
| | Width (mm) | 4 |
| | Thickness (mm) | 3 |
| | Shape grades (% rounds) 1: <20; 2: 20-40; | 2 |
| | 3: 40-60; 4: 60-80; 5: >80 | |
| | Pericarp color (dark purple) | 9 |
| | Aleurone color 1: homozygous; | 1 |
| | 2: segregating | |
| | Color 1: white; 2: pink; 3: tan; 4: brown; | 7 |
| | 5: bronze; 6: red; 7: purple, 8: palepurple; | |
| | 9: varigated | |
| | Endosperm color 1: white; 2: pale- | 1-2 |
| | yellow; 3: yellow; 4: pink-orange; | |
| | 5: white cap | |
| | Endosperm type 1: sweet; 2: extra sweet; | 3 |
| | 3: normal starch; 4: high amylose starch; | |
| | 5: waxy starch; 6: high protein; 7: high | |
| | lysine; 8: other | |
| | Weight/100 gm seed (unsized sample) | 12 |
| 9 | Cob | |
| | Diameter at midpoint (mm) | 24 |
| | Strength 1: weak; 2: strong | 2 |
| | Color 1: white; 2: pink; 3: red; 4: brown; | 6 |
| | 5: varigated; 6: other(dark purple) | |
| 10 | Disease resistance | |
| | Stalk rot(diplodia) | 0 |
| | Northern leaf blight | 1 |
| | Southern rust | 0 |
| | Bacterial leaf blight | 0 |
| | Anthracnose | 0 |
| | Stalk rot(fusarium) | 0 |
| | Southern leaf blight | 0 |
| | Corn smut | 0 |
| | Maize dwarf mosaic | 0 |
| | Stalk rot (gibberella) | 0 |
| | Smut | 0 |
| | Bacterial wilt | 0 |
| | Stunt | 0 |
| 11 | Insect resistance | |
| | Cornborer | 1 |
| | Rootworm(northern) | 1 |
| | Rootworm(southern) | 0 |
| | Rootworm(western) | 1 |
| | Earworm | 1 |
| 12 | Heat units | 8 |

The data presented in Tables 1 represent morphological traits associated with the Table of Characteristics published by the International Union for the Protection of New Varieties of Plants (UPOV). The data for Table 1 were gathered from observations in fields near Brookings S. Dak. and Lamberton Minn. The data represent ratings of a person of skill in the art without resorting to the reference inbreds specified in the UPOV publication Guidelines for the Conduct of Tests for Distinctness, Uniformity and Stability, TG/2/6, UPOV (1999). A more detailed description of these traits can be found in Guidelines for the Conduct of Tests for Distinctness, Uniformity and Stability, TG/2/6, UPOV (1999).

As can be seen in the rating data of Table 1, the inbred FAR614 exhibits characteristic unusual strong purple coloration in plant parts and tissue, including stems, stalks, leaves, sheaths, grains and husks. Novel traits characteristic of the invention are for instance intense purple coloration of the husks and stalks of the plant. Results of the extraction shown in the table 3 of the following pages confirm these ratings.

One aspect of the invention provides novel corn inbred FAR614 as purple-pigmented corn inbred with superior characteristics providing excellent male and/or female parental lines for producing F₁ corn hybrids also having excellent characteristics. It will be appreciated that the invention is intended to cover both inbred and hybrid plants and parts (tissues or cells) thereof. This includes any plant parts and tissues acceptable for use in extracting pigments present therein as well as for other uses described herein.

### Working Example 1: Observations and Anthocyanin Extraction from Plant Parts

Early versions of FAR614 were discovered in a planting containing multiple inbreds and hybrids in approximately 2004. Very little developmental work other than homozygous line improvement was conducted over the next many years.

Winter 2013 one nursery row used to make hybrid for testing & in line development.
Summer 2013 eight nursery rows used to make hybrids for testing & in line development.
Winter 2014 two nursery rows used to make hybrids for testing.
Summer 2014 ten nursery rows used to make hybrids for testing, in line development & for selfing to identify high anthocyanin segregates.
Further development is continuing.
Observations from past two years:
appears to be quite homozygous
low level of green tassel off types
has a high level of anthocyanins expression in the whole plant, especially in the stalk, tassel, husk & grain
medium short plant height
average plant health
husk opens at the tip
fairly small ear
flowers 8 days earlier than FAR601

Tissues from representative plants were sampled to determine anthocyanin concentrations extracted from each tissue. The plants were harvested near the black layer stage of maturity and characterized according to the following method:

Extraction of Anthocyanin from Corn Fractions:

The following corn fractions were evaluated: corn husks, corn grain (whole kernels) and corn stalks (stalks include all of the material from the stalk except for the tassels, leaves and root system)

1-Husks

Cut husks with scissors into approximately ¼ inch pieces. Blend husks using kitchen blender on ‘grate’ setting for 60 seconds to grind into smaller pieces. Blend in multiple batches. Grind husks further using coffee grinder for 30 seconds. Run in smaller batches and combine to make a composite. Husks may be ground finer with more suitable equipment. See photo 1 for example of ground material.

### Procedure:

Husk to water ratio was 8:92 (40 g husks+460 g deionized water) using 1 L Pyrex glass bottles. Place bottles tightly capped in heated reciprocating water bath (large unit). Place enough water in the unit to cover the bottle's surface.
Extraction temperature −75° C. 90 RPM—60 min extraction.
Coarse filter first through cheesecloth/dish towel over colander. Press and squeeze to remove as much extract as possible from the husks. Recovery may be improved with more suitable equipment.
Vacuum filter using VWR 415 filter paper. Record filtrate recovered.

2-Grain

Grain was evaluated as whole kernels without grinding in small scale evaluation tests.

### Procedure:

Grain was used as whole kernels (no grinding). Kernel to water ratio was 33:67 (5 g kernels to 10 g deionized water) using 40 ml glass vials. Place glass vials in wire basket and place in heated reciprocating water bath. Place enough water in the unit to cover the bottle's surface.
Extraction temperature −75° C. 90 RPM—60 min extraction.
Gravity filter through VWR 415 filter paper

3-Stalks

Stalks were cut into smaller pieces and roughly ground in kitchen blender for 60 sec on ‘grate’ setting. Ground stalks were then dried in pans for 2 days in small oven (40° C.) to dry completely. After this oven drying step, grind stalks in kitchen blender on ‘grate’ setting for 60 sec followed by coffee grinder for 30 sec, until a fine material is obtained (see picture 3). Stalks may be ground finer to a smaller particle size with more suitable equipment.

### Procedure:

Stalk to water ratio was 8:92 (4 g stalks, 46 g deionized water) using 125 mL Nalgene bottles. Place bottles tightly capped in heated reciprocating water bath (large unit). Place enough water in the unit to cover the bottle's surface.
Extraction temperature −75° C. 90 RPM—60 min extraction.
Coarse filter first through cheesecloth/dish towel over colander. Press and squeeze to remove as much extract as possible from the stalks. Recovery may be improved with more suitable equipment.
Vacuum filter using VWR 415 filter paper. Record filtrate recovered.

Buffer Preparation and Monomeric Anthocyanin Measurements:

Procedure, calculations and buffer preparation based on “Giusti and Wrolstad, 2001 Characterization and Measurement of Anthocyanins by UV-Visible Spectroscopy.”

External pH 3 Citrate Buffer Preparation—

Use in monomeric anthocyanin calculations.

### Prepare According to the Following:

1. Weigh 39.0 g Citric Acid Monohydrate in a 1000 mL volumetric flask.
2. Weigh 12.7 g Sodium Citrate Dihydrate in the same 1000 mL flask.
3. Fill to volume using DI water. Mix well until completely dissolved.
4. Check pH of solution using pH meter. Adjust with 50:50 citric acid (usually about 2.8 mL/L) and fill to the 1000 mL line. Mix well and measure pH to remain at 3.0+/−0.01.

Husk, Grain and Stalks—Monomeric Anthocyanin Measurements with External pH3 Citrate Buffer

Measure both sample filtrate and external pH3 citrate buffer by weight to the 0.0001 accuracy. Prepare dilutions to obtain an absorbance reading between 0.200-2.000. Run in duplicate dilutions and combine to measure average. Run wavelength scan (400-800 nm) on Evolution 60 Spectrometer/Colorimeter to obtain absorbance at peak wavelength and corrected for haze @ 700 nm. See example below:

Calculation were adjusted and based on “Giusti and Wrolstad, 2001 Characterization and Measurement of Anthocyanins by UV-Visible Spectroscopy”. The modification was the use of a single buffer (pH 3 citrate) instead of a dual buffer pH differential system to calculate monomeric anthocyanin. The use of pH 3 for the calculation likely underestimate monomeric anthocyanin values.
Calculate the absorbance of the diluted sample (A) as follows:

A=(Aλvis-max−A700)_{pH 1.0}−(Aλvis-max−A700)_{pH 4.5} Giusti and Wrolstad's procedure

A=(Aλvis-max−A700)_{pH 3.0} Suntava's calculations
Calculate the monomeric anthocyanin pigment concentration in the original sample using the following formula:

Monomeric anthocyanin pigment (mg/liter)=(A×MW×DF×1000)/(ε×1)

where MW is the molecular weight, DF is the dilution factor (for example, if a 0.2 ml sample is diluted to 3 ml, DF=15), and ε is the molar absorptivity.

“IMPORTANT NOTE: The MW and ε used in this formula correspond to the predominant anthocyanin in the sample. Use the ε reported in the literature for the anthocyanin pigment in acidic aqueous solvent. If the ε of the major pigment is not available, or if the sample composition is unknown, calculate pigment content as cyanidin-3-glucoside, where MW=449.2 and ε=26,900 (see Background Information, discussion of Molar Absorptivity). The equation presented above assumes a path length of 1 cm.”

### Working Example 2: Anthocyanin Extraction Results from Plants Harvested the Afton Location

As can be noted from table 3, FAR614 shows unusually high anthocyanin content in all plant parts and in particular in the glumes, husks and sheaths when compared to other hybrids such as FAR601 which is known for kernel coloration.

The inbred FAR614 was grown and crossed with inbred 1 near lamberton Minnesota in 2013 to produce the hybrid (inbred 1 by FAR614).

The results are shown in Table 4 where one can see that inbred 1, a comparative variety, lacks any significant coloration.

As can be seen in tables 4, FAR614 not only shows significantly higher levels of anthocyanins in the different plant parts particularly in the husks and stalks but also effectively transmits its high anthocyanin characteristic when hybridized with inbred 1 which, as can be seen, lacks any coloration in the husks and stalks. FAR601 however, although it shows some coloration of the kernel, it lacks any significant coloration in the other parts of the plant and it fails to impart any coloration to the husks and stalks.

### Working Example 3

Single Nucleotide Polymorphism (SNP) genotyping has become one of the most favored methods for characterizing the DNA of plants. See for instance: Martin Ganal et al. A Large Maize (Zea mays L.) SNP Genotyping Array: Development and Germplasm Genotyping, and Genetic Mapping to Compare with the B73 Reference Genome, Plosone, Vol. 6, Issue 2, 2011 and tabassum Jihan et al. Single Nucleotide Polymorphism, Method and Application in Plant Genetics: A Review. Department of Botany, Delhi University, Delhi 110 007, India

Inbreds FAR614 and a comparative inbred FAR601, known for high anthocyanin content, were assayed by BioDiagnostics, Inc., River Falls, Wis., for SNP genotype using methods described in the article cited above. A copy of the SNP genotyping results has been submitted with the provisional No. 62/093,580, Docket number 001US005, filed on Dec. 18, 2014 the disclosure of which is herein incorporated by reference in its entirety

There were 35,397 markers were investigated by the SNP method on the inbreds FAR601 and FAR614. 29,829 of the SNP markers were similar between the two inbreds. A total of 5,568 markers (SNP) were different between the two inbreds. Table shows a short example of the complete table submitted with the provisional of this application to the USPTO.

### DEPOSITS

Applicant has made a deposit of at least 2500 seeds of Inbred FAR614 with the American Type Culture Collection (ATCC), Manassas, Va. 20110 USA, ATCC Deposit Number: PTA-121874. The seeds deposited with the ATCC on Jan. 12, 2015 were taken from the deposit maintained by Suntava, Inc. 3290, Saint Croix Trail, P.O Box 268, Afton, Minn. 55001.

Access to these deposits will be available during the pendency of the application to the Commissioner of Patents and Trademarks and persons determined by the Commissioner to be entitled thereto upon request. Upon allowance of any claims in the application, the Applicant will make the deposit available to the public pursuant to 37 C.F.R. §1.808. These deposits of the Inbred FAR614 will be maintained in the ATCC depository, which is a public depository, for a period of 30 years, or 5 years after the most recent request, or for the enforceable life of the patent, whichever is longer, and will be replaced if either becomes nonviable during that period. Additionally, Applicant has satisfied all the requirements of 37 C.F.R. §§1.801-1.809, including providing an indication of the viability of the samples upon deposit. Applicant has no authority to waive any restrictions imposed by law on the transfer of biological material or its transportation in commerce. Applicant does not waive any infringement of Applicant's rights granted under this patent.

This invention has been described herein in considerable detail in order to comply with the patent statutes and to provide those skilled in the art with the information needed to apply the novel principles and to construct and use embodiments of the example as required. However, it is to be understood that the invention can be carried out by specifically different devices and that various modifications can be accomplished without departing from the scope of the invention itself.

What is claimed is:

1. A seed of a corn cultivar designated FAR614, representative seed of said cultivar deposited under ATCC Accession Number PTA-121874.

2. A corn plant, or a regenerable part thereof, produced by growing the seed of claim 1.

3. A tissue culture of regenerable cells produced from the plant of claim 2.

4. A protoplast produced from the tissue culture of claim 3.

5. The tissue culture of claim 3, wherein cells of the tissue culture are from a plant part selected from the group consisting of a leaf, a pollen grain, an embryo, a root tip, an anther, an inflorescence, a seed, a stem and a stalk.

6. A corn plant regenerated from the tissue culture of claim 3, said plant having all the morphological and physiological characteristics of corn cultivar FAR614, representative of said cultivar deposited under ATCC Accession Number PTA-121874.

7. A method for producing a hybrid corn seed wherein the method comprises crossing the plant of claim 2 with a different corn plant and harvesting the resultant hybrid corn seed.

8. A hybrid corn seed produced by the method of claim 7.

9. A hybrid corn plant produced by growing the hybrid seed of claim 8.

10. A method of producing an herbicide resistant corn plant wherein the method comprises transforming the corn plant of claim 2 with a transgene that confers herbicide resistance.

11. An herbicide resistant corn plant produced by the method of claim 10.

12. The corn plant of claim 11, wherein the transgene confers resistance to an herbicide selected from the group consisting of imidazolinone, sulfonylurea, glyphosate, glufosinate, L-phosphinothricin, triazine and benzonitrile.

13. A method of producing an insect resistant corn plant wherein the method comprises transforming the corn plant of claim 2 with a transgene that confers insect resistance.

14. An insect resistant corn plant produced by the method of claim 13.

15. The corn plant of claim 14 wherein the transgene encodes a Bacillus thuringiensis endotoxin.

16. A method of producing a disease resistant corn plant wherein the method comprises transforming the corn plant of claim 2 with a transgene that confers disease resistance.

17. A disease resistant corn plant produced by the method of claim 16.

18. A method of introducing a desired trait into corn cultivar FAR614 wherein the method comprises: crossing FAR614 plants grown from FAR614 seed, representative seed of which has been deposited under ATCC Accession Number PTA-121874, with plants of another corn line that comprise a desired trait to produce progeny plants, wherein the desired trait is selected from the group consisting of male sterility, herbicide resistance, insect resistance and disease resistance; selecting progeny plants that have the desired trait to produce selected progeny plants; crossing the selected progeny plants with the FAR614 plants to produce backcross progeny plants; selecting for backcross progeny plants that have the desired trait and physiological and morphological characteristics of corn cultivar FAR614 to produce selected backcross progeny plants; and repeating said crossing and selecting for backcross progeny plants steps at least three times in succession, or, when using molecular markers, repeating said crossing and selecting at least once and selfing said backcross progeny plants at least once, to produce respective selected fourth or higher backcross progeny plants or second-selfed backcross progeny plants that comprise the desired trait and physiological and morphological characteristics of corn cultivar FAR614.

19. A plant produced by the method of claim 18, wherein the plant has the desired trait and physiological and morphological characteristics of corn cultivar FAR614 as determined at the 5% significance level when grown in the same environmental conditions.

20. The plant of claim 19 wherein the desired trait is herbicide resistance and the resistance is conferred to an herbicide selected from the group consisting of imidazolinone, sulfonylurea, glyphosate, glufosinate, L-phosphinothricin, triazine and benzonitrile.

21. The plant of claim 19 wherein the desired trait is insect resistance and the insect resistance is conferred by a transgene encoding at least a portion of a Bacillus thuringiensis endotoxin.

22. A process of developing a corn variety, comprising sequentially inbreeding segregating generations of a corn hybrid having the plant of claim 2 as a parent until an advanced generation is attained, said advanced generation being F5 or greater and developing the corn variety.

23. The process of claim 22, in which inbreeding includes self-pollination.

24. A corn plant according to anyone of the preceding claims having anthocyanin amounts in any of its parts higher than 1 mg per gram of raw material.

25. An anthocyanin extract from the plant obtained according to anyone of the preceding claims.
